(19) United States
(12) Patent Application Publication
(10) Pub. No.: US 2006/0247581 A1
(43) Pub. Date: Nov. 2, 2006

(54) Threaded rod and nut assembly

(75) Inventors: Thomas Pedersen, Helsingoer (DK); Henrik Rasmussen, Lyngby (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/356,678

(22) Filed: Feb. 17, 2006

Related U.S. Application Data
(63) Continuation of application No. PCT/DK04/00583, filed on Sep. 2, 2004.

(30) Foreign Application Priority Data
Sep. 8, 2003 (DK) PA 2003 01292

(51) Int. Cl. A61M 5/315

(52) U.S. Cl. 604/218

(57) ABSTRACT

Disclosed is a stop for preventing a threaded rod from screwing thru a nut. The stop is formed by manufacturing a threaded rod having an unthreaded cylindrical portion and one or more grooves cut or manufactured longitudinally in the rod across both the threaded section and the unthreaded portion. A nut having an interior thread complementary to that of the rod has a hole bored thru its wall over a peak in the interior thread. The hole creates a stop surface on the interior thread that abuts a stop surface created by the groove in the unthreaded portion of the rod.

### FIELD OF THE INVENTION

The present invention relates to threaded rod and nut assemblies. In particular it relates to a stop means for preventing relative movement of the rod with respect to the nut. This is particularly relevant for injection devices, such as pen style syringes, which often use threaded piston rods and nuts to drive preset doses from the syringe.

### DESCRIPTION OF RELATED ART

A typical injection device comprises a threaded piston rod and a nut. Usually, the piston rod has a groove or grooves machined so that it has a not round cross-section. In general, two types of devices are common. In one type, a drive nut is coupled to a drive means that spins the drive nut, which is rotatable but is fixed in the housing to prevent longitudinal movement. An end-of-content mechanism for such device is disclosed in U.S. Pat. No. 6,582,404. In other devices, the nut is screwed along the piston rod during a dose setting operation. See e.g. US Patent Application Nos. 20020052578 A1, which is hereby incorporated by reference. The nut is screwed a linear distance along the piston rod from an abutment in the housing of the device. The distance the nut is moved linearly is the amount by which a piston rod may be displaced and thus relates to a specific quantity of medication that will be expelled from medication containing cartridge within the device. Regardless of the type of device, it is often desirable or necessary to create a stop on the piston rod so that the nut cannot be screwed off one end of the rod.

In devices where the nut is screwed along the piston rod during dose setting, when the stop is located at a predefined distance, it is possible to configure a device so that a dose may never be set that exceeds the quantity of medication remaining in the cartridge. Typically, stops have been constructed by ending the thread on the piston rod and having a structure with a larger diameter than the threaded portion abut the nut. This is disclosed in FIG. 1 in US 20020052578 A1 (the piston rod having reference No. 4, the unthreaded part having No. 35 and the nut having No. 13). However, when large torque loads are exerted on the piston rod and nut, the nut can jam. It would therefore be desirable to construct a nut and threaded rod assembly with a stop means that prevents the nut from being screwed off the piston rod without jamming.

### SUMMARY OF THE INVENTION

The present invention provides for a threaded rod and nut assembly having a stop means that prevents the nut from being screwed off the piston rod, at least in one direction. And it further provides for a method of making a nut and threaded rod assembly having a stop means. In one embodiment, a portion of a rod is threaded. Near a proximal end of the rod, the thread ends (preferably abruptly), and beyond that (in a proximal direction) there is a cylindrical surface having the same (or smaller) outside diameter as the largest diameter of the threaded portion. A groove or a plurality of grooves are machined longitudinally in the rod. A nut is threaded on the inside with a corresponding female thread preferably running only partly through the nut. A hole or opening is bored in the nut near a proximal end. The hole cuts into the end of the female thread and creates a stop surface within the nut. When the nut is screwed onto the rod, starting with the distal end of the nut, the nut screws along the rod in a distal direction until the groove in the cylindrical portion enters the region of the nut where the hole is bored. At this point, the surface in the cylindrical portion formed by the groove hits a thread on the interior of the nut and prevents relative rotation between the rod and the nut.

Thus, in one embodiment the present invention comprises a threaded rod having a portion of it outer surface threaded, a cylindrical portion disposed beyond where the threading ends, one or more grooves or flat surfaces machined in the outer surface, the grooves, extending to cover the length of the threaded portion and at least a portion of the cylindrical surface, thereby forming a stop surface on the rod, a nut having a threaded interior and a hole in the nut, thereby creating a stop surface in the nut for the stop surface of the nut to abut when the nut rod is screwed through the nut. Preferably, the hole is made over a peak (i.e., it should cover at least one peak) in the interior thread so that a cross-section of the peak of the thread forms the stop surface. Of course the hole may be larger than the peak to peak distance of the interior thread. This will result in the hole straddling at least one peak in an interior thread.

The above described embodiment may be created by a method comprising:

cutting a thread on the outer surface of a rod

removing the thread before the end of the rod, preferably abruptly, and

boring a hole in a nut, preferably such that the hole is not disposed between threads in a valley, but is disposed over a peak (or crest) of the thread.

### BRIEF DESCRIPTION OF THE DRAWINGS

The invention will be explained more fully in connection with a preferred embodiment and with reference to the drawings in which:

FIG. 1 shows a nut and threaded rod with the stop assembly of the present invention.

FIG. 2 shows a nut use with the assembly shown in FIG. 1.

FIG. 3 shows a threaded rod with a left handed pitch for use with the assembly shown in FIG. 1.

### DETAILED DESCRIPTION OF THE INVENTION

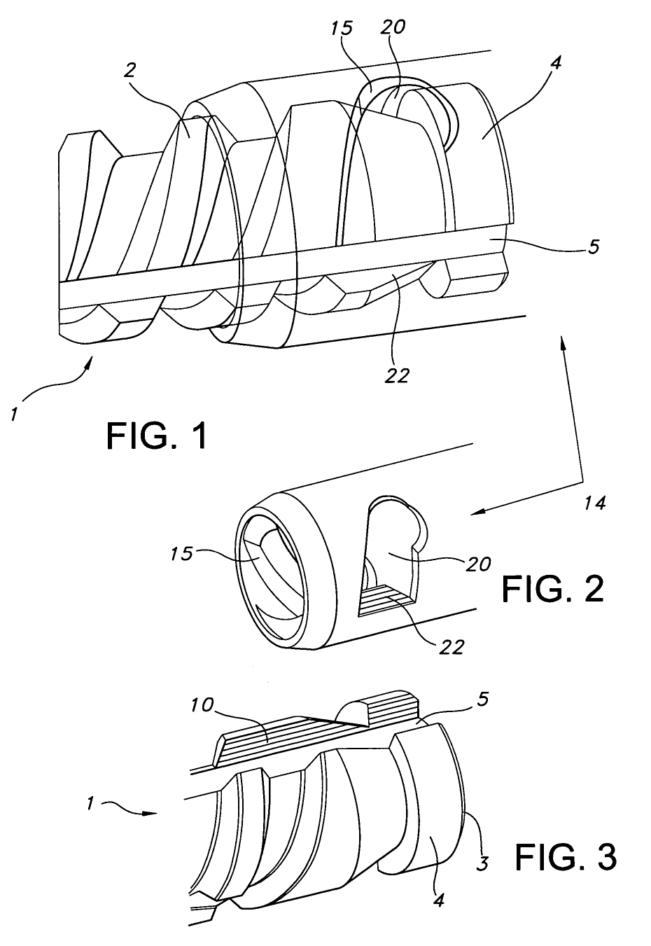

As is shown in FIG. 1, a rod 1, such as a piston rod for an injection device, is threaded with male threads 2. The threads 2 end prior to the end 3 of the rod 1, thereby leaving a cylindrical portion 4 that is unthreaded. One or more grooves 5 are machined into the rod 1 and run in a longitudinal direction. As shown in FIG. 3, the groove 5 cuts across the threads 2 and the cylindrical portion 4. This results in a stop surface 10 being formed on the cylindrical portion 4. Preferably the threads are cut with a cutting tool that is abruptly removed from the rod so that the thread ends abruptly. Moreover, the outer diameter of the cylindrical portion is preferably less than or equal to the maximum outer diameter of the threaded portion. The order of steps is not critical as long as the steps result in the above described structure. For example, the rod may be grooved or flat surfaces cut into it, before it is threaded. Of course, other methods besides cutting may be employed to create the threads and grooves.

A nut 14 having interior female threads 15 is modified to have a stop surface 22 that abuts the stop surface 10 on the rod 1. As shown in FIG. 2, a nut 14 having interior female threads 15 has a piece of material removed to form a hole 20. Preferably, the hole 20 is bored over a peak in a thread 15. This result in an interior stop surface 22 being formed in the nut 14. When the rod 1 is screwed through the nut 14, as is shown in FIG. 1, the stop surface 10 of the rod 1 will abut the stop surface 22 in the nut 14 and stop the rod 1 from rotating and linearly progressing through the nut 14. Since the outer diameter of the cylindrical portion of the threaded rod 1 may be smaller than or equal to the maximum diameter of the threaded portion of the rod 1, the only significant contact upon stopping are the two stop surfaces 10, 22. The stop surfaces 10, 22 contact each other with virtually no binding and as a result the rod may be rotated in an opposite direction with little effort. The stop surfaces 10, 22 resist binding even when high torque loads are exerted on the nut 14 and rod 1.

The above described invention is broad in scope and should not be limited to the embodiments disclosed herein. While it has been described as useful in injection devices having threaded piston rods, it is usefully in other applications where it is desirable to stop a nut from rotating off a threaded rod or to stop a rod from rotating through a nut. Furthermore, it is useful in end of content devices for injection devices such as that described in U.S. Pat. No. 6,582,404 and other devices where a stop is needed for a nut that screws along a threaded shaft, barrel, rod or other surface.

Some preferred embodiments have been shown in the foregoing, but it should be stressed that the invention is not limited to these, but may be embodied in other ways within the subject matter defined in the following claims.

1. A threaded rod and nut assembly with a stop that prevents the nut (14) from screwing off the rod (1) in one direction, the assembly comprising:
a rod (1) having a thread (2) disposed on a portion of its outer surface, a cylindrical portion (4) of the rod (1) remaining unthreaded, and
a nut (14) having a female thread (15) on a at least a portion of its inner surface,
Characterized in that, the peak of the female thread (15) ends in a stop surface (22) within the nut (14).

2. A threaded rod and nut assembly according to claim 1, characterized in that, a hole (20) is provided over the peak in the interior thread (15) of the nut (14) from the inner surface to the outer surface thereby forming the stop surface (22) within the nut (14).

3. A threaded rod and nut assembly according to claim 1, characterized in that, the peak of the female thread (15) ends abruptly.

4. A threaded rod and nut assembly according to claim 3, characterized in that, the stop surface (22) within the nut (14) is substantially parallel with the longitudinal axis of the nut (14).

5. A threaded rod and nut assembly according to any of the preceding claims, characterized in that, one or more grooves (5) in a longitudinal direction is provided in the rod (1).

6. A threaded rod and nut assembly according to claim 5, characterized in that, the grooves (5) extends through the threaded area and into the cylindrical unthreaded portion (4), thereby forming a rod stop surface (10) on the cylindrical portion (4)

7. A threaded rod and nut assembly according to claim 5, characterized in that the rod (1) has at least two grooves (5) preferably opposite each other.

8. A threaded rod and nut assembly according to anyone of the claims 5, characterized in that, the rod stop surface (10) is substantially parallel with the longitudinal axis of the rod (1).

9. A threaded rod and nut assembly according to anyone of the preceding claims, characterized in that, the stop surface (22) within the nut (14) and the rod stop surface (10) interfaces each other in the stop position.

10. A method of manufacturing a threaded rod (1) and nut (14) assembly with a stop that prevents the nut (14) from screwing off the rod (1) in one direction according to any of the claims 1 to 8, the method comprising the steps of:
i) forming a thread (2) on the outer portion of a cylindrical rod (1),
ii) stopping the threading prior to reaching an end of the rod (1), thereby leaving a cylindrical portion (4) of the rod (1) unthreaded,
iii) forming one or more longitudinal grooves (5) in at least a portion of the outer surface of the rod (1), thereby forming a stop surface (10) on the rod (1), and
iiii) creating a hole (20) in a threaded nut (14) at least partly through a raised portion (peak) of a thread (15) on the interior of the nut (14), thereby forming a complimentary stop surface (22) within the nut (14).

11. The method of claim 10, characterized in that, the grooves (5) are formed in the rod (1) prior to the thread (2) being created.

12. The method of claim 11, characterized in that, a thread cutting tool machines the thread (2) into the rod (1) and is abruptly removed from the rod (1).
